(19) United States
(12) Patent Application Publication
(10) Pub. No.: US 2014/0046098 A1
(43) Pub. Date: Feb. 13, 2014

(54) Catalytic Conversion Of Alcohols To Aldehydes Or Ketones

(71) Applicant: Melvin K. Carter, Lincoln, CA (US)

(72) Inventor: Melvin K. Carter, Lincoln, CA (US)

(73) Assignee: Carter Technologies, Lincoln, CA (US)

(21) Appl. No.: 13/570,208

(22) Filed: Aug. 8, 2012

(51) Int. Cl. C07C 45/38; C07C 47/07; C07C 49/00; C07C 49/10; C07C 49/08; C07C 45/39; C07C 47/02

(52) U.S. Cl. 568/402

(57) ABSTRACT

Catalytic reactions are taught using air or oxygen for oxidative chemical conversion of primary alcohols to aldehydes and for secondary alcohols to ketones in a vapor phase at ambient pressure. The catalytic process converts ethanol to acetaldehyde, n-propanol to propionaldehyde, 2-propanol to acetone, and other alcohols to aldehydes and ketones. The catalysts are based on molecular strings of di-, tri- and/or poly-groups of transition metal complexes possessing a specific degree of symmetry. Laboratory results have demonstrated [vanadium (II)]₂, [chromium (II)]₂, [manganese (II)]₂, [cobalt (II)]₂ oxalate and symmetric transition metal catalysts to be effective for oxidative catalytic conversion of primary alcohols to products comprising related aldehydes and secondary alcohols to products comprising ketones.

### BACKGROUND

1. Field of Invention

Catalytic chemical processes have been reported for formation of aldehydes and ketones by partial dehydrogenation of alcohols, by hydration of alkynes, by controlled air oxidation of hydrocarbon gases and as a by-product from industrial fermentation processes. Controlled air oxidation of certain gaseous hydrocarbons or alcohols may produce small amounts of aldehyde, however such processes have not been identified as economically viable. The invention disclosed in this application teaches catalytic oxidative conversion of alcohols with air or oxygen selectively to aldehydes and ketones using mono-metal, di-metal, tri-metal and/or poly-metal backbone or molecular string type transition metal catalysts possessing a specific degree of symmetry without addition of aggressive chemical oxidizing agents and without addition of other strong chemicals.

2. Description of Prior Art

The chemical process industry has grown to maturity based on petroleum feed stocks. Petroleum is a non-renewable resource that may become unavailable in the next 100 to 150 years. This planet Earth fosters continual growth of numerous carbohydrate based plants including fruits, vegetables and grain food sources plus their supporting cellulosic plant stalks and related natural waste materials for recycle. Grains, corn cobs, the support plant stalks and certain grasses are subject to catalytic conversion processes and, in part, subject to bio-fermentation processes producing ethanol and other alcohol products. A major industry is rapidly developing in ethanol production by catalytic and fermentation of bio-mass and much of the product is sold as combustion engine fuel or its additive. Ethanol is becoming more available as a renewable resource and this application teaches its catalytic conversion to valued intermediates for use in production of hydrocarbon fuels and industrial chemicals.

A number of catalytic chemical processes have been reported converting alcohols to aldehydes as presented here. Acetaldehyde has been produced by hydration of acetylene, by controlled air oxidation of propane and butane from natural gas, and as a by-product from the fermentation industries. It can also be produced by reduction of acetic acid and by carbonylation of methanol. Controlled air oxidation of certain gaseous hydrocarbons may produce small amounts of aldehydes, however such processes have not been identified as economically viable. Acetaldehyde formation by direct air oxidation of ethanol has not previously been productive.

There are several hot tube reactions described in the scientific and patent literature for conversion of gaseous alcohols to a wide range of low concentration products from gasoline type hydrocarbons to aldehydes and ethers. Aldehydes and ketones can be formed by passing alcohol vapors over Cu and its alloys or Ag at 300C to 600C in the presence of controlled amounts of air. U.S. Pat. No. 6,166,265, issued Dec. 26, 2000, introduced a process for preparation of n-butyraldehyde and/or n-butanol by reacting butadiene with an alcohol at super-atmospheric pressure and elevated temperatures using an acid resin or one of several transition metal oxides. U.S. Pat. No. 6,350,918, issued Feb. 26, 2002, teaches a process for the selective oxidation of alcohols to aldehydes at 150C to 600C over oxides of V, Cr, Mo, W or Re in their high oxidation states. Less selective chemistry may oxidize an alcohol to aldehydes, ketones and by products. Aldehydes have also been produced by a chemical exchange where one oxidized organic compound may transfer its oxygen atoms to an alcohol converting it to an aldehyde.

Acetaldehyde has also been produced commercially by oxidation of ethanol in air at 480C and super-atmospheric pressure in the presence of a silver catalyst. This has been replaced by the Wacker process for oxidation of ethylene that has been more efficient than previous ethanol oxidation methods. Both processes start with ethylene. Acetaldehyde has also been produced by the expensive hydration of acetylene on a mercury salt catalyst process. Acetaldehyde can be produced from synthesis gas using a rhodium metal on silica catalyst at elevated temperature and pressure, but the selectivity to acetaldehyde is poor. Acetaldehyde has also been produced by reacting methanol with synthesis gas at elevated temperature and pressure using a cobalt iodide catalyst with a promoter, however neither the rhodium metal nor cobalt iodide catalytic process has been practiced commercially. U.S. Pat. No. 6,465,694, issued Oct. 15, 2002, reported conversion of polyethylene glycol to aldehyde derivatives in the presence of potassium carbonate over a Cu, Co, Fe, Ni metal catalyst in air at 40C to 90C but un-derivatized aldehyde was not reported. U.S. Pat. No. 6,121,498, issued Sep. 19, 2000, disclosed reduction of carboxylic acid compounds to their respective aldehydes in hydrogen gas over palladium on iron oxide catalyst at temperatures of 250C to 400C. U.S. Pat. No. 5,679,870, issued Oct. 21, 1997, published conversion of ketene in hydrogen gas over Co, Rh, Ir, Ni, Pd or Pt metal catalyst to aldehydes in a temperature range of 50C to 200C. U.S. Pat. No. 4,351,908, issued Sep. 28, 1982, reported carbonylation from synthesis gas on a rhodium metal catalyst at 75C to 125C to form alcohols and aldehydes.

Ketones can be prepared by oxidation of secondary alcohols. The process normally requires a strong oxidizing agent such as potassium permanganate or potassium dichromate. The alcohol is oxidized by heating under reflux in acidified solution. For example 2-propanol is oxidized to propanone (acetone) where two atoms of hydrogen are removed for each molecule leaving a carbon-oxygen double-bond. U.S. Pat No. 4,453,015, issued Jun. 5, 1984, disclosed catalytic conversion of secondary butanol to methyl ethyl ketone over a copper, zinc and chromium metal catalyst on an alpha alumina support at 1 to over 65 atmospheres pressure and 250C to 450C. No reports were identified disclosing catalytic oxidative conversion of alcohols to aldehydes or ketones at ambient pressure without strong chemical additives.

The invention disclosed in this application teaches oxidative catalytic conversion of alcohols with air or oxygen to aldehydes and ketones using mono-metal, di-metal, tri-metal and/or poly-metal backbone or molecular string type transition metal catalysts in a low oxidation state possessing a specific degree of symmetry without addition of zero valent metals, oxides, silica, aggressive chemical oxidizing agents and without addition of other strong chemicals. This catalytic process results in majority yields of the reported products.

It is an object of this invention, therefore, to provide a molecular string type transition metal catalytic process for air or oxygen oxidative conversion of primary alcohols to aldehydes without the use of aggressive chemical oxidizing agents or other strong chemicals.

It is another object of this invention to provide molecular string type catalysts for direct air or oxygen oxidation of secondary alcohols to ketones without the use of aggressive chemical oxidizing agents or other strong chemicals. Other objects of this invention will be apparent from the detailed description thereof that follows, and from the claims.

### SUMMARY OF THE INVENTION

This invention describes catalytic chemical oxidative processes for conversion of alcohols in air or oxygen at ambient pressure to aldehydes or ketones using transition metal catalysts based primarily on di-metal, tri-metal and/or poly-metal backbone or strings possessing a specific degree of symmetry.

### DETAILED DESCRIPTION OF THE INVENTION

A process is taught for catalytic chemical oxidative conversion of primary alcohols to aldehydes and secondary alcohols to ketones in the presence of air or oxygen employing specific transition metal compounds, such as [vanadium (C₂H₂O₄)]₂, [manganese (C₂H₂O₄)]₂ or [cobalt (C₂H₂O₄)]₂ type compounds, for which the transition metals and directly attached atoms possess C₄ᵥ, D_{4b} or D_{2d} point group symmetry. These catalysts have been designed based on a formal theory of catalysis, and the catalysts have been produced, and tested to prove their activity. The theory of catalysis rests upon a requirement that a catalyst possess a single metal atom or a molecular string such that transitions from one molecular electronic configuration to another be barrier free so reactants may proceed freely to products as driven by thermodynamic considerations. Catalysts effective for chemical conversion of alcohols to aldehydes or ketones can be made from mono-metal, di-metal, tri-metal and/or poly-metal backbone or molecular string type compounds of the transition metals comprising titanium, vanadium, chromium, manganese, iron, cobalt, nickel, copper, zirconium, niobium, molybdenum, ruthenium, rhodium, palladium, silver, hafnium, tantalum, tungsten, rhenium, osmium, iridium, platinum, gold and/or combinations thereof. These catalysts are made in the absence of oxygen so as to produce compounds wherein the oxidation state of the transition metal is low, typically monovalent or divalent although trivalent metal catalysts may also be produced. Anions employed for these catalysts comprise fluoride, chloride, bromide, iodide, cyanide, isocyanate, thiocyanate, sulfate, phosphate, borate, oxalate, acetate and organic chelating agents. Mixed transition metal compounds have also been found to be effective catalysts for oxidative chemical conversions.

The catalysts act on primary alcohols in the presence of air or oxygen effectively removing terminal hydrogen atoms forming aldehydes under conditions of relatively low temperature and a relatively high air flow rate. Secondary alcohols are catalytically oxidized to ketones under most conditions at somewhat higher temperatures. For example ethanol is catalytically oxidized to acetaldehyde using a [cobalt (II) oxalate]₂ catalyst in a temperature range of 125C to 200C while n-propanol is catalytically converted to propionaldehyde under similar conditions. By contrast 2-propanol is catalytically converted to acetone at temperatures of 150C to 200C.

### DESCRIPTION OF CATALYST PREPARATIONS AND CHEMICAL CONVERSIONS

Catalyst preparation was conducted using nitrogen purging and/or nitrogen blanketing to minimize or eliminate air oxidation of the transition metal compounds during preparation. Transition metal catalysts, effective for ambient pressure conversion of alcohols and similar hydroxy substituted organic compounds, can be produced by combining transition metal salts in their lowest standard oxidation states with anionic salts. Thus, such transition metal catalysts can be made by reacting transition metal (I or II) acetate, chloride, bromide, iodide, sulfate, phosphate, borate or cyanide compounds with other transition metal (I or H) compounds or chelates, or by forming transition metal compounds in a reduced state where mono-, di-, tri- and/or poly-metal compounds result. Some examples follow.

### Example 1

The Co₂(C₂H₂O₄)₂ catalyst was prepared in a nitrogen atmosphere by addition of 0.249 gram of cobalt (II) acetate, dissolved in 3 mL of nitrogen purged water, to 15 grams of ⅛ inch diameter alumina silicate cylinders and evaporating to dryness. To this was added 0.433 gram of potassium hydrogen oxalate, dissolved in 15 mL of nitrogen purged water, and the resultant catalyst was heated to approximately 125C until dry.

### Example 2

The Mn₂(C₂H₂O₄)₂ catalyst was prepared in a nitrogen atmosphere by addition of 0.0989 gram of manganese (II) chloride, dissolved in 3 mL of nitrogen purged water, to 15 grams of ⅛ inch diameter alumina silicate cylinders and evaporating to dryness. To this was added 0.216 gram of potassium hydrogen oxalate, dissolved in 15 mL of nitrogen purged water, and the resultant catalyst was heated to approximately 125C until dry.

Chemical conversion to aldehydes or ketones was conducted as described. The catalyst was loaded into a stainless steel tube reactor and maintained at its operating temperature. Air was supplied by means of a gas pump, its flow rate was monitored by a gas flow meter, ethanol was delivered by means of a syringe pump and injected onto the catalyst vaporizing immediately. Resulting vapor products were collected using a cold trap and identified by means of a wet chemical indicator. Ethanol was injected at a rate of 0.20 mL/minute and air was supplied at rates of 0.20 L/minute to 1 L/minute during the reactions at temperatures in the range of 125C to 200C.

### Example A

Air was supplied at a rate of 1 L/minute to a cobalt oxalate catalyst in a reactor controlled at a temperature of 125C while ethanol was supplied at a rate of 0.20 mL/minute. A majority of acetaldehyde was produced. Air was also supplied at a rate of 1 L/minute to a cobalt oxalate catalyst controlled at temperatures of 150C and 175C while ethanol was supplied at a rate of 0.20 mL/minute. Again a majority of acetaldehyde was produced.

### Example B

Air was supplied at a rate of 0.8 L/minute to a cobalt oxalate catalyst in a reactor controlled at a temperature of 130C while ethanol was supplied at a rate of 0.20 mL/minute. A majority of acetaldehyde was produced. Air was also supplied at a rate of 0.8 L/minute to a cobalt oxalate catalyst controlled at temperatures of 150C and 190C to 200C with ethanol supplied at a rate of 0.20 mL/minute. Again a majority of acetaldehyde was produced. The same process was conducted at an air flow rate of 0.67 L/minute and catalyst temperatures of 135C, 175C and 202C producing a majority of acetaldehyde.

### Example C

Air was supplied at rates of 0.20 L/minute, 0.35 L/minute and 0.50 L/minute to a cobalt oxalate catalyst in a reactor controlled at a temperature of 125C while ethanol was supplied at a rate of 0.20 mL/minute. A majority of acetaldehyde was produced. Again air was supplied at rates of 0.20, 0.35 and 0.50 L/minute to a cobalt oxalate catalyst controlled at temperatures of 162C and 200C while ethanol was supplied at a rate of 0.20 mL/minute. A mixture of acetaldehyde and ethoxyethanol was produced.

### Example D

Isopropyl alcohol was injected on to a cobalt oxalate on silica-alumina catalyst packed tube reactor in the temperature range of 175C to 200C at air flow rates of 0.20, 0.35 and 0.50 L/minute where acetone was produced.

What was claimed:

1a. Catalytic chemical oxidative conversion of ethyl alcohol in a vapor phase with air or oxygen to acetaldehyde on a non-oxide transition metal compound comprising [vanadium (II)]₂, [chromium (II)]₂, [manganese (II)]₂ or [cobalt (II)]₂ oxalate.

2a. Catalytic chemical oxidative conversion of ethyl alcohol in a vapor phase with air or oxygen to acetaldehyde on a non-oxide transition metal compound comprising [vanadium (II)]₂, [chromium (II)]₂, [manganese (II)]₂ or [cobalt (II)]₂ oxalate based on selected mono-metal, di-metal, tri-metal and/or poly-metal backbone or molecular string type complexes possessing a degree of symmetry being in C₄ᵥ, D_{2d} or D₄ₕ point group molecular symmetry configuration, formed from transition metal compounds in a low oxidation state of 2+ comprising titanium, vanadium, chromium, manganese, iron, cobalt, nickel, zirconium, niobium, molybdenum, ruthenium, rhodium, palladium, silver, hafnium, tantalum, tungsten, rhenium, osmium, iridium, platinum, gold or combinations thereof at ambient pressure and temperatures in the range of 125C to 200C.

3a. Catalytic chemical oxidative conversion of isopropyl alcohol in a vapor phase with air or oxygen to propanone (acetone) on a non-oxide transition metal compound comprising [vanadium (II)]₂, [chromium (II)]₂, [manganese (II)]₂ or [cobalt (II)]₂ oxalate.

4a. Catalytic chemical oxidative conversion of isopropyl alcohol in a vapor phase with air or oxygen to propanone (acetone) on a non-oxide transition metal compound comprising [vanadium (II)]₂, [chromium (II)]₂, [manganese (II)]₂ or [cobalt (II)]₂ oxalate based on selected mono-metal, di-metal, tri-metal and/or poly-metal backbone or molecular string type complexes possessing a degree of symmetry being in C₄ᵥ, D_{2d} or D₄ₕ point group molecular symmetry configuration, formed from transition metal compounds in a low oxidation state of 2+ comprising titanium, vanadium, chromium, manganese, iron, cobalt, nickel, zirconium, niobium, molybdenum, ruthenium, rhodium, palladium, silver, hafnium, tantalum, tungsten, rhenium, osmium, iridium, platinum, gold or combinations thereof at ambient pressure and temperatures in the range of 125C to 200C.

5a. Catalytic chemical oxidative conversion of primary alcohols in a vapor phase with air or oxygen to alkyl aldehydes comprising acetaldehyde, propyl aldehyde and butyl aldehyde (butyraldehyde) on a non-oxide transition metal compound comprising [vanadium (II)]₂, [chromium (II)]₂, [manganese (II)]₂ or [cobalt (II)]₂ oxalate.

6a. Catalytic chemical oxidative conversion of primary alcohols in a vapor phase with air or oxygen to alkyl aldehydes comprising acetaldehyde, propyl aldehyde and butyl aldehyde (butyraldehyde) on a non-oxide transition metal compound comprising [vanadium (II)]₂, [chromium (II)]₂, [manganese (II)]₂ or [cobalt (II)]₂ oxalate based on selected mono-metal, di-metal, tri-metal and/or poly-metal backbone or molecular string type complexes possessing a degree of symmetry being in C₄ᵥ, D_{2d}, or D₄ₕ point group molecular symmetry configuration, formed from transition metal compounds in a low oxidation state of 2+ comprising titanium, vanadium, chromium, manganese, iron, cobalt, nickel, zirconium, niobium, molybdenum, ruthenium, rhodium, palladium, silver, hafnium, tantalum, tungsten, rhenium, osmium, iridium, platinum, gold or combinations thereof at ambient pressure and temperatures in the range of 125C to 200C.

7a. Catalytic chemical oxidative conversion of secondary alcohols in a vapor phase with air or oxygen to ketones comprising propanone (acetone), butanone (methyl ethyl ketone) and pentanones on a non-oxide transition metal compound comprising [vanadium (II)]₂, [chromium (II)]₂, [manganese (II)]₂ or [cobalt (II)]₂ oxalate.

8a. Catalytic chemical oxidative conversion of secondary alcohols in a vapor phase with air or oxygen to ketones comprising propanone (acetone), butanone (methyl ethyl ketone) and pentanones on a non-oxide transition metal compound comprising [vanadium (II)]₂, [chromium (II)]₂, [manganese (II)]₂ or [cobalt (II)]₂ oxalate based on selected mono-metal, di-metal, tri-metal and/or poly-metal backbone or molecular string type complexes possessing a degree of symmetry being in C₄ᵥ, D_{2d} or D₄ₕ point group molecular symmetry configuration, formed from transition metal compounds in a low oxidation state of 2+ comprising titanium, vanadium, chromium, manganese, iron, cobalt, nickel, zirconium, niobium, molybdenum, ruthenium, rhodium, palladium, silver, hafnium, tantalum, tungsten, rhenium, osmium, iridium, platinum, gold or combinations thereof at ambient pressure and temperatures in the range of 125C to 200C.
